(19) United States
(12) Patent Application Publication
(10) Pub. No.: US 2009/0018547 A1
(43) Pub. Date: Jan. 15, 2009

(54) MINIMAL ACCESS OCCIPITAL DRILL/TAP PERSUADER

(75) Inventor: MATT CREWS, Memphis, TN (US)

Correspondence Address: RATNERPRESTIA, P.O. BOX 980, VALLEY FORGE, PA, 19482, US

(73) Assignee: AESCULAP IMPLANT SYSTEMS, INC., CENTER VALLEY, PA (US)

(21) Appl. No.: 12/147,971

(22) Filed: Jun. 27, 2008

Related U.S. Application Data
(60) Provisional application No. 60/958,893, filed on Jul. 10, 2007.

(51) Int. Cl. A61B 17/58

(52) U.S. Cl. 606/96

(57) ABSTRACT

An assembly includes a lever to guide a surgical tool, such as drill, during operation. The lever includes an arm for engaging a surgical tool. A guide member is pivotally coupled to the lever. The arm is pivotally displaceable relative to the guide member in an arc-shaped path, while the guide member is adapted to restrict movement of a surgical tool in a generally linear direction in response to pivotal displacement of the arm.

### RELATED APPLICATIONS

This non-provisional application claims priority to U.S. Provisional Application Ser. No. 60/958,893, filed Jul. 10, 2007, the entire contents of which are incorporated by reference herein for all purposes.

### FIELD OF THE INVENTION

The present invention relates generally to posterior fusion of the spine, and more specifically to instrumentation and techniques for drilling and tapping bone during posterior fusion.

### BACKGROUND OF THE INVENTION

During posterior fusion of the occipito-cervico-thoracic junction, fusion of the spine at, for example the OCT/T3 junction, requires the insertion of screws into the occiput bone at the rear of the skull. Drilling and tapping holes at the rear of the skull can be a difficult procedure which requires a significant amount force to penetrate the dense cortical bone, which is very hard. The drilling angle at the rear of the skull also creates a difficult approach angle for the surgeon. On top of these challenges, the drill depth must be controlled with precision, which is difficult to do while at the same time applying significant force on the drill at a difficult angle of approach.

### SUMMARY OF THE INVENTION

The competing objectives and technical challenges of posterior fusion at the OCT/T3 junction and other sections of the spine are resolved by assemblies for guiding surgical tools in accordance with the invention.

In a first aspect of the invention, an assembly for guiding surgical tools includes a persuader lever having an arm for engaging a surgical tool, and a guide member pivotally coupled to the persuader lever. The arm is pivotally displaceable relative to the guide member in an arc-shaped path, and the guide member is adapted to restrict movement of a surgical tool in a generally linear direction in response to pivotal displacement of the arm.

In a second aspect of the invention, an assembly for guiding instruments includes a lever having a gripping end with a pair of prongs. A guide member is pivotally coupled to the lever. One end of the guide member includes a first tool guide having a bore with a longitudinal axis. The prongs straddle the longitudinal axis of the bore.

In a third aspect of the invention, an assembly for guiding instruments includes a guide member having at least one tool guide, and a lever with a gripping end. The lever is pivotally coupled to the guide member such that the gripping end is confined to an arc-shaped range of motion relative to the guide member. The at least one tool guide includes a bore substantially aligned with the gripping end, and a stop that limits the range of motion of the gripping end relative to the guide member.

### BRIEF DESCRIPTION OF THE DRAWINGS

The foregoing summary and the following descriptions may be more clearly understood when read in conjunction with the drawing figures, of which:

FIG. 1 is a perspective illustration of the occiput bone region of the skull;

FIG. 2 is a perspective view of a partial model of the occiput bone region of the skull, shown during a first simulated procedure in accordance with one aspect of the invention;

FIG. 3 is a perspective view of a partial model of the occiput bone region of the skull, shown during a second simulated procedure in accordance with another aspect of the invention;

FIG. 4 is a perspective view of an exemplary embodiment of an assembly in accordance with another aspect of the present invention, shown with components of a surgical drill.

FIG. 5 is a perspective view of components of an exemplary drill that may be used in accordance with another aspect of the present invention;

FIG. 6 is an enlarged perspective view of a portion of the drill shown in FIG. 5;

FIG. 7 is an enlarged perspective view of a portion of the assembly and drill shown in FIG. 4;

FIG. 8 is an enlarged elevation view of a portion of the assembly and drill shown in FIG. 4;

FIG. 9 is a perspective view of an exemplary embodiment of a persuader lever in accordance with another aspect of the present invention;

FIG. 10 is a perspective view of an exemplary embodiment of a guide component in accordance with another aspect of the present invention;

FIGS. 11-13 are partial elevation views of an exemplary embodiment of an assembly and drill in accordance with another aspect of the invention, illustrating different stages of operation of the assembly;

FIG. 14 is a perspective view of exemplary embodiments of an assembly and tap bit in accordance with another aspect of the present invention; and

FIG. 15 is a perspective view of exemplary embodiments of a tool and tap bit in accordance with another aspect of the present invention.

### DETAILED DESCRIPTION OF PREFERRED EMBODIMENTS OF THE INVENTION

Although the invention is illustrated and described herein with reference to specific embodiments, the invention is not intended to be limited to the details shown. Rather, various modifications may be made in the details within the scope and range of equivalents of the claims and without departing from the invention.

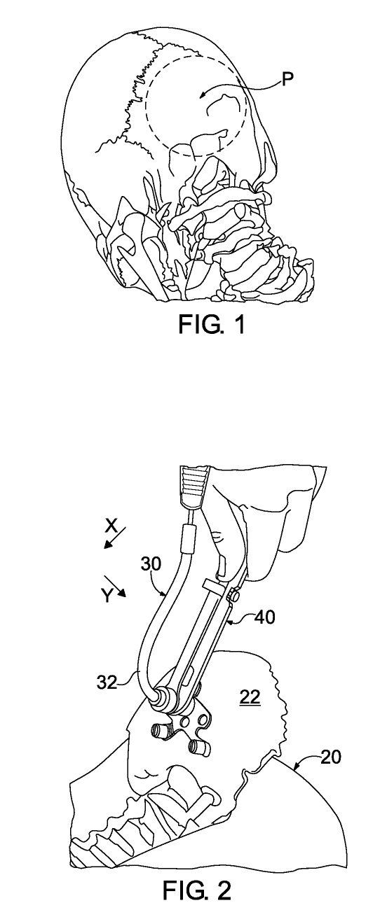

Applicant proposes an apparatus and procedure for posterior fusion of the occipito-cervico-thoracic junction of the spine. In the procedure, a plate is implanted onto the occiput bone of the skull. The approximate area of this portion of the skull is identified by the arrow labeled P in FIG. 1. The occiput bone is formed of hard cortical bone. To secure the plate to the bone, the applicant proposes to anchor one or more bone screws into the bone. Holes for the screws are prepared by initially drilling a small hole into the bone, and subsequently tapping the hole with a threaded tap bit. As the tap bit is driven into the hole, the thread on the tap bit carves a corresponding thread along the sidewall of the hole. The resulting thread inside the hole is configured to mate with a thread on a bone screw.

The surgeon will typically need to access the bone from an approach angle or direction that is different from the direction of drilling. That is, the direction of approach is angularly offset from, or transverse to, the drilling and tapping direction. To facilitate the change in direction, a flexible drill bit is employed, which allows the surgeon to operate the drill from a first direction and advance the drill bit in a second direction. Applying force in the first direction does not advance the drill bit in the second direction, however.

Applicant proposes using a drill/tap persuader to advance the operating drill bit or tap bit in the second direction. The drill/tap persuader is configured to advance the drill bit or tap bit in a controlled path aligned with the desired drilling direction.

Referring to FIG. 2, a simulated drilling procedure is shown with instrumentation and techniques in accordance with one example of the invention. A model 20 includes a cortical bone 22 which is exposed for purposes of illustration. A drill 30 with a flexible drill bit 32 is operated to drill a hole into the cortical bone 22. The angle of approach, which is represented by arrow X, is transverse to the angle of advancement of the drill, which is represented by arrow Y. Because drill bit 32 is flexible, force applied in direction X is not effective in advancing the drill bit in direction Y. Therefore, to advance drill bit 32 in direction Y, a persuader assembly 40 is connected with drill 30 to advance drill bit 32 in the desired drilling direction Y. The surgeon can operate persuader assembly 40 with one hand, leaving the other hand free to operate the drill 30.

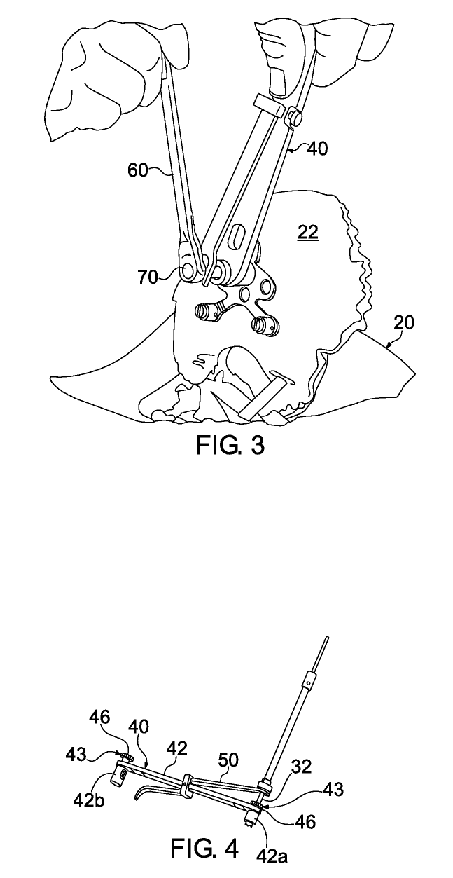

Persuader assembly 40 is also operable to guide and advance a tap bit into the drilled hole, after drilling is completed. In FIG. 3, persuader assembly 40 is shown in simulated operation with a ratchet tool 60 and tap bit 70. Persuader assembly 40 urges or guides the tap bit 70 into the drilled hole as torque is applied to the ratchet tool 60.

Referring to FIG. 4, the persuader assembly 40 includes a drill guide 42 and a persuader lever 50. The flexible drill bit 32 is inserted through drill guide 42 and engaged by the persuader lever 50. Drill guide 42 includes an elongated body with a first guide 42a at one end, and a second guide 42b at the opposite end. First and second guides 42a, 42b are hollow and each form a bore for receiving a drill bit. The bores of first and second guides 42a, 42b have different diameters to accommodate different sized drill bits. In the illustration, drill bit 32 is shown inserted in first guide 42a. It will be understood that a drill bit may be inserted though second guide 42b in cases where second guide 42b would be more appropriately sized for the drill bit.

Guides 42a, 42b each have an adjustable stop 43 that is axially displaceable in the guides. The stops 43 control the depth of advancement of the drill bit in the guides. Each stop 43 has a threaded exterior that engages an internal thread inside its respective guide. The stops 43 are rotatable within their respective guides to move axially with respect to the axis of the guide, which is aligned with the axis of the drill bit. A knurled dial 46 on each stop 43 permits the surgeon to rotate the stop and adjust the position of the stop to a desired setting that corresponds to a depth of insertion of a drill bit. The dial 46 provides an abutment that limits advancement of the drill bit 32, as described in more detail below.

Referring to FIG. 10, drill guide 42 is shown with guides 42a, 42b adjusted to different settings. The stop in guide 42b is adjusted to a higher position than the stop in guide 42a. In this arrangement, the drill depth is limited to a shallower depth when is guide 42b is used, as compared to guide 42a.

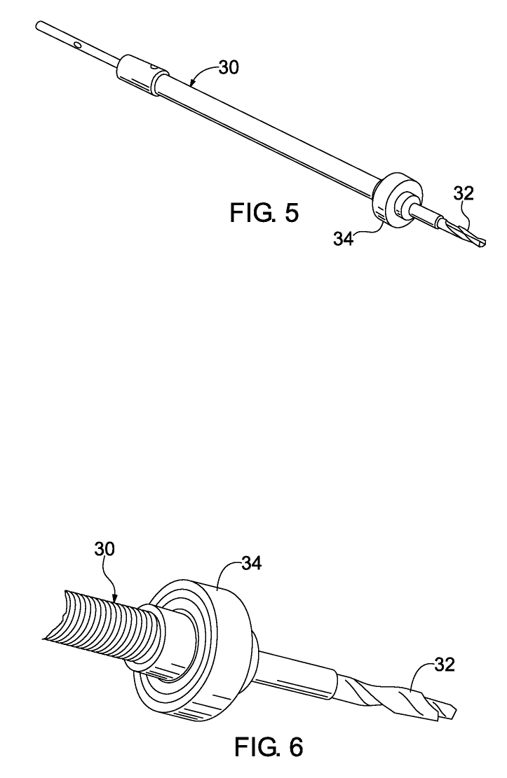

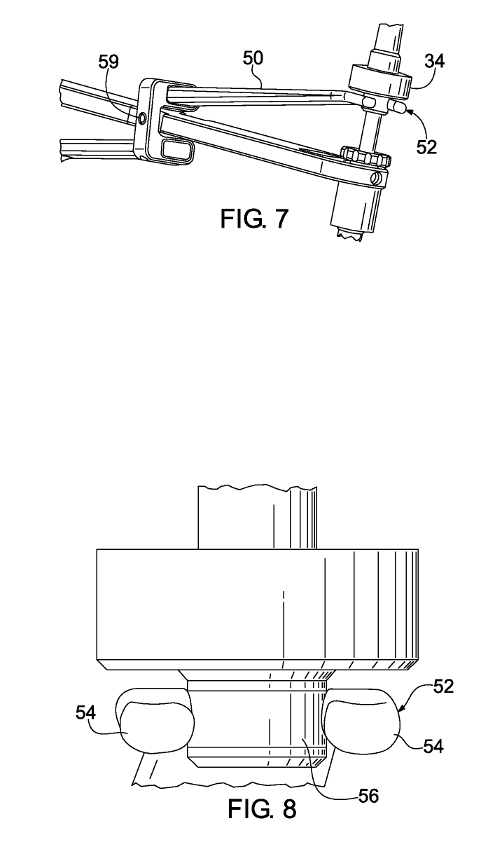

FIGS. 5 and 6 illustrate the flexible drill 30 and drill bit 32 in greater detail. It will be understood that various drills and bits may be used, and the selection is not limited merely to those shown. Drill 30 includes a bearing 34 that is gripped by the persuader assembly 40 during operation. Referring to FIGS. 7 and 8, the bearing 34 of drill 30 is gripped by persuader lever 50. Persuader lever 50 has a forked end 52 with prongs 54. The prongs 54 are separated by a gap or channel 56 adapted to receive a narrow portion beneath the bearing 34 of drill 30. When the persuader lever 50 grips the bearing 34, the lever is operable to advance the drill bit 32 relative to the guide in which the bit is inserted.

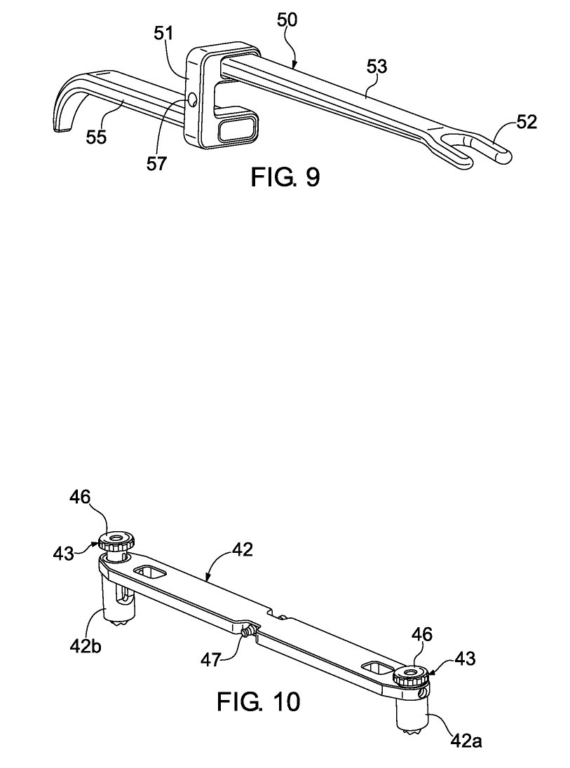

Referring now to FIGS. 9 and 10, the persuader lever 50 and drill guide 42 will be described in greater detail. Persuader lever 50 has a forked end 52 that is supported on an arm 53. A central hub 51 separates arm 53 from a handle portion 55 that extends on the opposite side of the hub. Hub 51 includes a central aperture 57 that extends through the body of the hub. Drill guide 42 includes a pin 47 located generally at the center of the drill guide. Central aperture 57 on the persuader lever 50 pivotally connects with the pin 47, forming a hinge connection 59 that is shown best in FIG. 7. When the handle portion 55 is pivoted toward drill guide 42 (i.e. “raised”), the arm 53 and forked end 52 will pivot toward the drill guide. Similarly, when the handle portion 55 is pivoted away from drill guide 42 (i.e. “lowered”), the arm 53 and forked end 52 will also pivot away from the drill guide. When persuader assembly 40 is connected with a drill bit 32, the act of raising the handle will advance the drill bit through the guide, and lowering the handle will reverse the drill bit out of the guide.

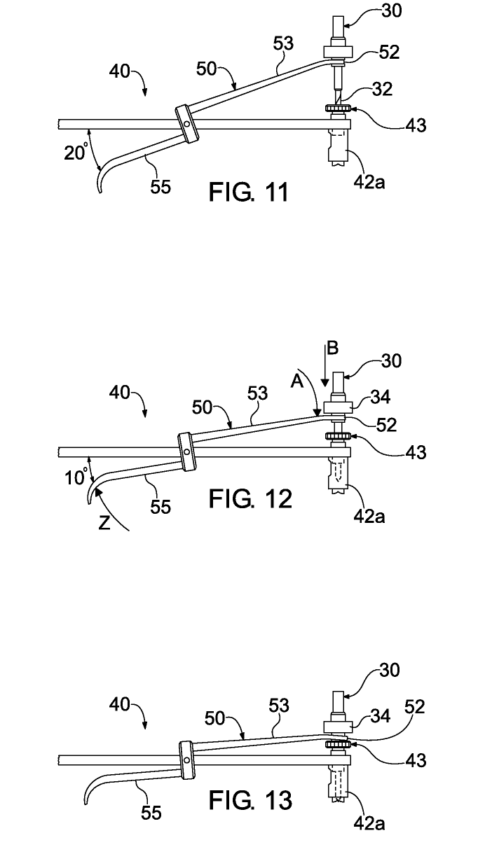

Referring now to FIGS. 11-13, the operation of the persuader assembly 40 to insert a drill bit is illustrated. In FIG. 11, handle 55 is shown in a lowered position, at an angle of approximately 20 degrees from the drill guide 42. In this position, the drill bit is positioned in a withdrawn or “pre-insertion” position. Because the handle 55 is lowered, the arm 53 and forked end 52 are in a raised position with respect to the guide 42a. Forked end 53 engages the drill 30 at a position beneath the bearing 34. The axial position of drill bit 32 is controlled by the position of the forked end 52.

Referring now to FIG. 12, handle 55 is raised from its position in FIG. 11 and moved in the direction shown by arrow Z. That is, handle 55 is moved toward the guide 42 to an angle of approximately 10 degrees with respect to the guide. This displacement may be carried out by squeezing the handle upwardly or toward the guide 42. In response, the arm 53 and forked end 52 are pivoted toward the drill guide 42a, with the arm forming an angle of approximately 10 degrees with respect to the guide. Movement of the forked end 52 persuades the drill bit toward a “partially-inserted” position. Although the arm 53 pivots in an arc-shaped motion about the hinge connection 59, as shown by arrow A, motion of the drill bit 32 is limited by drill guide 42a to a tangential direction, as shown by arrow B. The drill guide 42a limits motion of drill bit 32 so that the drill bit only moves in the desired drilling direction. As the forked end 53 travels through the arc-shaped path, the prongs tilt slightly relative to the axis of the drill bit, allowing the bit to move tangentially without binding inside the guide 42a.

FIG. 13 shows the handle 55 is a fully raised position, and the drill bit 32 in a “fully-inserted” position. In this position, the forked end 52 of arm 53 abuts the stop 43, preventing further advancement of the drill bit 32, and consequently further raising of the handle 55. The axial position of the stop 43, as adjusted and set by the knurled dial 46, corresponds to a predetermined depth of insertion for the drill bit.

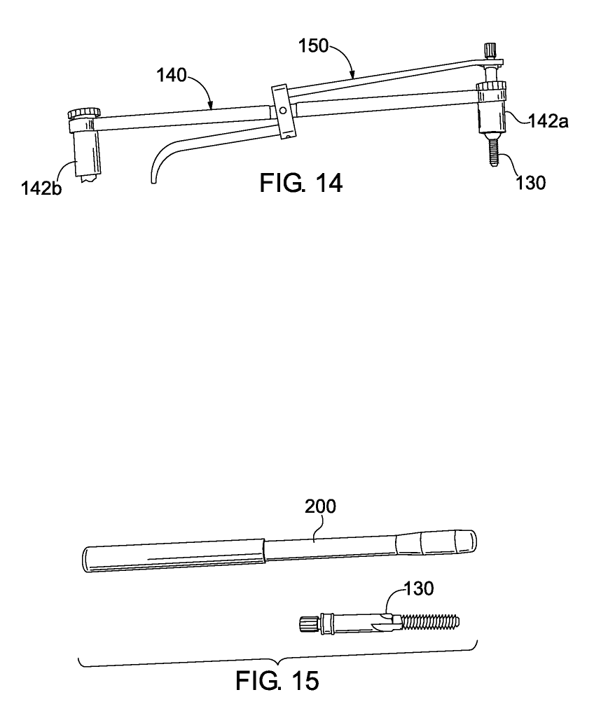

As noted above, the persuader assembly of the present invention is compatible with both drill guides and tap guides. Referring now to FIG. 14, an alternative embodiment of a persuader assembly 140 in accordance with the invention is shown with a tap bit 130. Persuader assembly 140 is similar in many respects to persuader assembly 40. Therefore, only distinctions will be discussed with the understanding that the operation and function of persuader assembly 140 are essentially identical to the operation and function of persuader assembly 40. A tap guide 142 includes first and second guides 142a, 142b that are adapted to receive and guide axial movement of the tap bit 130. A persuader lever 150 is operable to advance and reverse the tap bit during ratcheting of the tap bit. Movement of the tap bit is essentially restricted to rotational and linear movement, with linear motion being aligned with the axis of the drill hole being tapped. FIG. 15 illustrates the tap bit 130 in greater detail, and a ratcheting tool 200 that may be used to drive the tap bit into the pre-drilled hole.

The term “persuasion” as used herein shall refer to any form of manipulation of surgical instruments or accessories, including but not limited to advancement of implements such as flexible drills and screw taps. Thus far, instruments and methods for instrument persuasion during posterior fusion of the occipito-cervico-thoracic junction fusion of the spine have been described. These instruments and methods may be used, for example, at the OCT/T3 junction of the occiput to the vertebrae. The present invention is not limited solely to these specific procedures or areas of the body, however. Instruments and methods in accordance with the invention may be used for other surgical techniques where direct or indirect access to the surgical site is difficult, such as Odontoid screw fixation or accetabular cup fixation, to name just two examples.

Various embodiments and features are contemplated in accordance with the present invention, which may exist in combination or separately. The persuader may be compatible with both drill and tap guides at variable depths between 6-16 mm. This is merely a range of common depths, however. Drilling and tapping depths are dependent on patient anatomy and the specific drilling or tapping location. Therefore, the persuader may be compatible with other drill depths outside the range of 6-16 mm. The persuader may be removable if it is not necessary in the minimal access anatomy.

The persuader is compatible with various diameter drills and taps. Moreover, the persuader can be used in other drilling and tapping procedures, including procedures for drilling holes in other areas of the anatomy that present dense cortical bone being drilled at a difficult angle.

The persuader enables the surgeon to drill and tap the occiput bone with minimal wound length in the superior/inferior direction. In addition, the persuader provides a mechanical advantage that allows the surgeon to penetrate and tap harder areas of bone, such as cortical bone which is typical in the occiput bone portion of the skull. For example, the persuader can be used to apply additional axial force on the bit of a flexible drill or other surgical implement, providing the surgeon with the axial force needed to penetrate the bone. The persuader assembly provides for a one-handed operation, so that the surgeon is free to use the other hand to operate the drill, tap or other implement being assisted by the persuader assembly.

In further embodiments of the invention, the assembly may include adjustable drill and tap guides to allow for variable depths to be achieved with using only one instrument to either drill or tap. The persuader may have full stroke for all depths. Moreover, the persuader can allow insertion force and longitudinal progression of the drill and tap while allowing rotational motion (i.e. translation of rotational torque in to linear motion). Where flexible drills are used, the flexible shaft can allow translation of rotational motion at several angles, including angles greater than 20 degrees. The bearing housing or Teflon spacer allows the drill to free spin while making fixed contact to the persuader arm allowing normal force translation. The persuader allows the surgeon to actively control the drill insertion pressure while minimizing drill and tap guide pressure on the occipital bone.

The persuader maintains a tangential trajectory on the drill and tap with the use of geometrical relationships of translating point contacts. This allows the persuader to interconnect with the tap and drill guide and translate motion normal to the drill guide. The tangential point of contact is maintained through full motion of the persuader.

While preferred embodiments of the invention have been shown and described herein, it will be understood that such embodiments are provided by way of example only. Numerous variations, changes and substitutions will occur to those skilled in the art without departing from the spirit of the invention. Accordingly, it is intended that the appended claims cover all such variations as fall within the spirit and scope of the invention.

What is claimed:

1. An assembly for guiding surgical tools, the assembly comprising:
a persuader lever having an arm for engaging a surgical tool; and
a guide member pivotally coupled to the persuader lever,
wherein the arm is pivotally displaceable relative to the guide member in an arc-shaped path, and the guide member is adapted to restrict movement of the surgical tool in a generally linear direction in response to pivotal displacement of the arm.

2. The assembly of claim 1, wherein the arm comprises a plurality of prongs.

3. The assembly of claim 1, wherein the guide member comprises a pivot member at a midsection of the guide member, the persuader lever being interconnected to the guide member by the pivot member.

4. The assembly of claim 1, wherein the guide member comprises a first end and a second end opposite the first end, the first end comprising a first tool guide, the first tool guide comprising a bore with a bore diameter for receiving a tool.

5. The assembly of claim 4, wherein the second end comprises a second tool guide comprising a bore for receiving a tool, the bore for the second tool guide having a bore diameter that is different from the bore diameter of the first tool guide.

6. The assembly of claim 1, wherein the guide member comprises a stop positioned in the arc-shaped path of the arm, the stop arranged to abut the arm and limit the arm's range of motion relative to the guide member.

7. An assembly for guiding instruments, the assembly comprising:
a lever having a gripping end, the gripping end comprising a pair of prongs;
an guide member pivotally coupled to the lever; and
a first tool guide at one end of the guide member, the first tool guide comprising a bore having a longitudinal axis, the prongs being positioned to straddle the longitudinal axis of the bore.

8. The assembly of claim 7, wherein the guide member comprises a pivot member at a midsection of the guide member, the lever being interconnected to the guide member by the pivot member.

9. The assembly of claim 7, wherein the guide member comprises a first end and a second end opposite the first end, the first end comprising the first tool guide, and the second end comprising a second tool guide.

10. The assembly of claim 9, wherein the second tool guide comprises a bore, the bores of the first and second tool guides having different diameters.

11. The assembly of claim 7, wherein the guide member comprises a stop positioned in the arc-shaped path of the arm, the stop arranged to abut the arm and limit the arm's range of motion relative to the guide member.

12. The assembly of claim 11, wherein the guide member comprises a tool guide having a bore, and the stop is axially displaceable in the bore to change the arm's range of motion relative to the guide member.

13. An assembly for guiding instruments, the assembly comprising:
a guide member having at least one tool guide; and
a lever having a gripping end, the lever pivotally coupled to the guide member such that the gripping end is confined to an arc-shaped range of motion relative to the guide member,
the at least one tool guide comprising a bore substantially aligned with the gripping end, and a stop that limits the range of motion of the gripping end relative to the guide member.

14. The assembly of claim 13, wherein the guide member comprises a pivot member at a midsection of the guide member, the lever being interconnected to the guide member by the pivot member.

15. The assembly of claim 13, wherein the guide member comprises a first end and a second end opposite the first end, the at least one tool guide comprising a first tool guide at the first end and a second tool guide at the second end.

16. The assembly of claim 15, wherein the first and second tool guides each comprise a bore and a bore diameter, the bore diameter of the first tool guide being different from the bore diameter of the second tool guide.

17. The assembly of claim 16, wherein at least one of the tool guides comprises a stop positioned in the arc-shaped path of the gripping end, the stop arranged to abut the gripping end and limit the gripping end's range of motion relative to the guide member.

18. The assembly of claim 17, wherein the stop extendable with respect to the tool guide to change the gripping end's range of motion relative to the guide member.

19. The assembly of claim 16, wherein each of the tool guides comprises a stop operable to abut the gripping end and limit the gripping end's range of motion relative to the guide member.

20. The assembly of claim 15, wherein the guide member comprises a pivot member at a center point along the guide member, such that the guide member is connectable with the lever in either a first position in which the first tool guide is aligned with the gripping end, or a second position in which the second tool guide is aligned with the gripping end.
